(19) United States
(12) Patent Application Publication
(10) Pub. No.: US 2021/0016781 A1
(43) Pub. Date: Jan. 21, 2021

(54) SYSTEMS AND METHODS FOR A VEHICLE

(71) Applicant: VISTEON GLOBAL TECHNOLOGIES, INC., Van Buren Township, MI (US)

(72) Inventors: Hari-hara-sudhan Karunai-Ramanujam, Canton, MI (US); John William Whikehart, Northville, MI (US); Michael K. Ingrody, Canton, MI (US); Satish Putta, Van Buren Township, MI (US)

(21) Appl. No.: 16/516,739

(22) Filed: Jul. 19, 2019

(51) Int. Cl. B60W 40/08; B60Q 5/00; G05D 1/00; B60Q 1/52; B60W 30/18; A61B 5/18; A61B 5/0205; A61B 5/16; A61B 5/00; A61B 5/11; H04W 4/40

(52) U.S. Cl. CPC B60W 40/08; A61B 5/024; G05D 1/0088; B60Q 1/52; B60W 30/18; A61B 5/18; A61B 5/0205; A61B 5/163; A61B 5/4809; A61B 5/1116; A61B 5/747; A61B 5/74; A61B 5/7405; H04W 4/40; B60W 2040/0818; B60W 2540/26; G05D 2201/0213; B60Q 5/005

(57) ABSTRACT

A system for a vehicle for determining a capability of a person, such as an occupant of the vehicle. The system may include a sensor for monitoring the person. The system may include a processor coupled to the sensor. The processor may determine the capability of the person. The processor may communicate an indicator, from the system to a remote device, based on the capability of the person. The processor may operate the vehicle in an autonomous driving mode of operation. The processor may operate the vehicle in the autonomous driving mode of operation when the capability of the person is below a threshold value.

### BACKGROUND

### Field

One or more embodiments described herein relate to a vehicle for transmitting and/or receiving health information of an occupant of the vehicle or an occupant of another vehicle, such as a surrounding vehicle. The vehicle includes an autonomous mode of operation. As an example, the vehicle may serve as an emergency vehicle, which may operate in the autonomous mode of operation.

### Related Art

During operation of a vehicle, a driver of the vehicle may encounter a health issue, which may impair the driver's ability to operate the vehicle. For example, the health issue may impair the driver's vision, which may make road navigation impractical or impossible. As another example, the health issue may decrease the driver's reaction time. The health issue may result from a medical condition or another reason. When experiencing the health issue, the driver may have to wait for emergency personnel to arrive at the driver's location, before first-aid or other assistance may be rendered. When experiencing the health issue, such as in the case of a life-threatening emergency, time may be crucial to successfully address the health issue.

### SUMMARY

According to one or more embodiments, a method can include determining a driving capability of a driver of a vehicle while driving. The method can also include communicating an indicator of the capability of the driver from the vehicle.

In one or more embodiments, an apparatus can include at least one processor and at least one memory including computer program code. The at least one memory and the computer program code can be configured to, with the at least one processor, cause the apparatus at least to perform a process. The process can include determining a driving capability of a driver of a vehicle while driving. The process can also include communicating an indicator of the capability of the driver from the vehicle.

A non-transitory computer-readable medium can, in one or more embodiments, be encoded with instructions that, when executed in hardware, perform a process. Some, all, or none of the hardware may be in the vehicle, and some, all, or none of the hardware may be in a remote computing site, such as a cloud server. The process can include determining a driving capability of a driver of a vehicle while driving. The process can also include communicating an indicator of the capability of the driver from the vehicle. The process can further include converting the vehicle to an autonomous emergency vehicle.

One or more embodiments include a system for a vehicle. The system includes a processor and a memory. The memory includes computer program code. The memory and the computer program code, with the processor, are able to cause the system to determine a capability of a person and communicate an indicator, from the system to a remote device. The system may communicate the indicator based on the capability of the person.

One or more embodiments include a system for a vehicle. The system includes a sensor for monitoring an occupant of the vehicle and a processor coupled to the sensor. The processor, based on information from the sensor, may determine a condition of the occupant of the vehicle. The processor may determine, based on the condition, a capability of the occupant. The processor may communicate an indicator, from the system to a remote device outside of the vehicle, based on the capability of the occupant.

One or more embodiments include a non-transitory computer-readable medium. The non-transitory computer-readable medium includes instructions that, when executed by a processor, cause the processor to operate a vehicle in an autonomous mode of operation. This may be by performing the steps of: monitoring, via a sensor, an occupant of the vehicle; determining, based on the monitoring of the occupant, a condition of the occupant; determining, based on the condition of the occupant, a capability of the occupant; and operating the vehicle in the autonomous mode of operation, when the capability of the occupant is below a threshold value.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 generally illustrates a method according to the principles of the present disclosure.

FIG. 2 generally illustrates a system according to the principles of the present disclosure.

FIG. 3 generally illustrates a scenario according to the principles of the present disclosure.

### DETAILED DESCRIPTION

According to one or more embodiments, a method can include determining a capability, such a driving capability, of a person, such as a driver or other occupant, of a vehicle while in the vehicle (e.g., driving the vehicle or riding in the vehicle). The driving capability can be an attentiveness of the person or a sleep state of a person. The determining can include monitoring eye tracking, monitoring facial expressions, monitoring consciousness, monitoring pain, monitoring steering departures, monitoring an output of a heart rate monitor, monitoring an output of an electrocardiogram, monitoring blood pressure, monitoring person posture, or any combination thereof.

The method can also include communicating an indicator of the capability of the person from the vehicle, for example to an entity outside the driving vehicle. The communicating can include broadcasting or multicasting the capability. The communicating can include communicating to a plurality of nearby vehicles. The communicating may be performed using vehicle to everything (V2X), WiFi, or another wireless communication technology. The method can further include increasing an autonomous driving level of the vehicle based on the determination of the driving capability of the vehicle by the person. The method can also include an autonomous safe-stop.

The communicating can include communicating travel information regarding the vehicle. For example, the travel information can include speed of the vehicle, direction of the vehicle, acceleration of the vehicle, geographic location of the vehicle, or any combination of thereof. The communicating can further include communicating an autonomous mode status of the vehicle.

The communicating can include contacting an emergency responder. The emergency responder can include such people as firefighters, police, paramedics, ambulances, dispatchers, or the like. The method can also include contacting a health services provider regarding the person. For example, hospitals, emergency rooms, clinics, doctors, or the like can be contacted. The contacting can include informing an emergency room of a projected arrival time of the person. The contacting can also or alternatively include coordinating an exchange point with an ambulance to deliver the person to the ambulance. The method can include communicating a predicted traffic route of the vehicle to a point of exchange or hospital. This route can be provided to a system that can coordinate traffic signals or otherwise control traffic. The route can also be provided to other vehicles to give priority right of way to the vehicle.

The contacting the health services provider can include communicating health information of the person to the health services provider. The health information can include eye movement data, pain data, facial expression data, consciousness data, heart rate data, blood pressure data, electrocardiogram data, posture data, or any combination thereof.

The communicating can include emitting an audible alert, a visual alert, or a combination thereof, from the vehicle. This may convert an otherwise normal autonomous or semi-autonomous vehicle into an emergency vehicle.

The method can include communicating with an infrastructure system or law enforcement system to request permission to change traffic controls or break traffic controls. Changing the traffic controls can include changing a color of a traffic light. Breaking the traffic controls can include exceeding a posted speed limit.

The process can further include converting the vehicle to an autonomous vehicle, such as an autonomous emergency vehicle or other autonomous vehicle. In some embodiments, the process may determine whether the capability is above a threshold (e.g., indicating that the driver is not capable of driving the vehicle) and may convert the vehicle to the autonomous vehicle based on the determination that the capability is above the threshold. Additionally, the process can include automatically controlling autonomous vehicles in the projected traffic path to provide a clear path for the vehicle.

The above system and method can be variously implemented. For example, a vehicle, such as an autonomous vehicle (AV), may monitor an occupant's health or attentiveness. This may be through an occupant monitor device, which can include one or more sensors to monitor the occupant.

Various sensors can be used. For example, a bio-sensor may monitor heart-rate, blood pressure, electrocardiogram (EKG), and the like. This bio-sensor may, for example, be a smart watch or similar device. In another example, a camera may monitor a person's facial expressions and consciousness. An audio sensor may monitor pain.

A person's own vehicle can be referred to as an ego vehicle. The vehicle can monitor a person's active driving capability and attention to the vehicle. The vehicle can use an active driving monitoring eye-tracker or can use Bluetooth low energy (BLE) health monitoring profiles. If an abnormality is detected, the vehicle can broadcast, multicast, or otherwise transmit an indication of the abnormality to any or all nearby vehicles or structures using V2X, cellular, WiFi, or the like. The messages that are transmitted can include the vehicle's position, heading and speed. Other vehicles can use these messages to display a warning for drivers of those other vehicles to react to the driving path of the ego vehicle. If the other vehicles are in autonomous driving mode, the other vehicles can predict or otherwise calculate a way to navigate away from the vehicle's predicted path of travel.

The vehicle can also take an automatic route guidance to a nearby emergency care center or hospital. Additionally, the vehicle can provide an audible and/or visual alarm signal so as to convert itself into an emergency vehicle when such identification is needed.

The travel path of the vehicle and the projected travel time of the vehicle can be transmitted to a destination emergency center or hospital, which can permit timely preparation for medical attention or for a quicker medical intervention by reducing the overall travel time of an ambulance to reach the person in need.

The vehicle may communicate (for example, broadcast) to nearby vehicles and/or pedestrians that the vehicle is experiencing an emergency situation. The vehicle may request nearby vehicles and/or pedestrians to move out of the way, either with wireless radio communications, or using emergency lights, sounds, or the like to visually and/or audibly inform others of the emergency situation.

The vehicle may request permission from an emergency system to exceed speed limits, allow the vehicle to pass uninhibited, such as changing timing on stoplights, or the like. The vehicle may switch to an emergency response mode from a personal mode, private driver mode, chauffeur mode, or the like. The vehicle may request a route for navigating to a medical facility. The vehicle may ask what road entrance to use, what drop off point (for example, door) to use for the driver or other vehicle occupant.

The vehicle may receive driving commands from the medical facility. The medical facility may take control of driving functionality of the vehicle. There may be a handover to the medical facility once the vehicle is within a certain proximity or on a certain road. The medical facility may identify another vehicle for the vehicle to follow to the medical facility. The medical facility may broadcast or otherwise communicate to vehicles and pedestrians surrounding the medical facility to clear a path for the vehicle.

One or more embodiments may provide various benefits and/or advantages. For example, a vehicle, such as an autonomous vehicle (AV), may determine that an occupant (for example, the driver) is experiencing a health-issue. Alternatively, the occupant may inform the vehicle that the occupant is experiencing a health issue. One or more embodiments may advantageously reduce the time between this notification of a health issue and obtaining medical treatment. For example, one or more embodiments may autonomously switch the driving vehicle into an autonomous ambulance. Moreover, one or more embodiments may ensure that a receiving hospital or other medical facility is prepared in advance for the emergency medical care needed by the occupant.

The medical facility, first responder, or the like, may send commands to the vehicle to reduce the immediate health issue or make the person more comfortable, such as changing the air temperature inside the vehicle cabin or by allowing more air inside the vehicle (for example, by opening the windows or increasing a fan speed). The commands may also specify whether sharp turns and/or sudden accelerations/decelerations are permitted.

Additionally, one or more embodiments may reduce the time of a potential intercepting ambulance to reach the patient in need. Furthermore, one or more embodiments may reduce the possibility of an affected person or vehicle getting too close to another vehicle or pedestrian or otherwise being involved in a compromise of safety. One or more embodiments may further reduce the time to attend to a person's health emergency.

For example, one or more embodiments may transmit (for example, using a health beacon), the driver's or another occupant's health hazard condition to other vehicles to permit the other vehicles react accordingly. Moreover, one or more embodiments may autonomously and/or automatically switch a driving vehicle into an autonomous ambulance.

Additionally, one or more embodiments may ensure that a medical facility that is receiving the vehicle occupant is provided with early warning of the need for emergency medical care. Also, one or more embodiments may reduce the time of a potential intercepting ambulance, helicopter, boat, or other vehicle to reach the vehicle occupant.

In one or more embodiments, a system can broadcast health hazard information to all nearby vehicles and structures along with the host vehicle's position, heading, and speed, as well as hazard severity and type. A vehicle receiving such broadcast information can display A-HUD and/or cluster warnings about the nearby vehicle person's critical health stats. For privacy, the details of the health information may be obscured, but other drivers and/or vehicles may be warned that the person is not fully able to operate the vehicle. If the receiving vehicle is in autonomous driving mode, the received information can be used to predict ways to navigate away from a health hazard beaconing vehicle's predicted path of travel.

The vehicle itself can take an automatic route guidance to a nearby emergency room, urgent care, clinic, or hospital. Additionally, as mentioned above, the vehicle can provide visual and audible alarm signals, thereby identifying itself as an emergency vehicle. The vehicle can also transmit travel path, projected travel time, and occupant health information to the emergency destination.

Thus, for example, a vehicle using a driving monitoring system or a BLE health monitoring profile can detect that an emergency arises regarding the driver's health (or the health of a non-driver occupant). The driver monitoring system can internally communicate to a vehicle connectivity module, which can use V2X, WiFi, or any other desired subsystem.

The information from the health or driver monitoring system can be further processed by the connectivity module to continuously communicate (for example, broadcast, although individually addressed communication is also permitted) to external vehicles using V2X beacon messages, which contain critical health warning, positional information, speed and projected trajectory path of movement of the vehicle. This message, if received by a remote vehicle, can automatically cause the remote vehicle to display a warning message on any of the remote vehicle's driver display units for the remote vehicle's driver to react to the hazardous situation to deviate away from the predicted path of the vehicle. If the remote vehicle is equipped with an autonomous driving unit, the remote vehicle can drive itself out of the vehicle's predicated driving path. Optionally, the remote vehicle can further relay the emergency beacon information.

The vehicle, if equipped with autonomous driving capability, can also trigger the autonomous driving mode, search for a nearby emergency response center or hospital and drive the person to the hospital by itself. The projected time to reach the hospital, severity of medical care needed by the person can be transmitted to the hospital ahead of time, while traveling to the hospital. The severity can be indicated directly (for example, driver totally unresponsive, driver with limited responsiveness, or the like) or indirectly. Examples of indirect indications of severity may include measured heart rate, eye-movement, breathing pattern, or the like. In one or more embodiments, the car may attempt to determine a medical condition of the occupant as a preliminary medical assessment and may pass this preliminary medical assessment to the destination medical facility. For example, the vehicle may determine that the occupant's systems are consistent with intoxication, a stroke, a heart attack, or epilepsy.

The vehicle can also send audible and visual beacons similar to an ambulance, such that a nearby vehicle, which does not have either V2X or other autonomous driving capability can clear the way for the vehicle to proceed as an emergency vehicle.

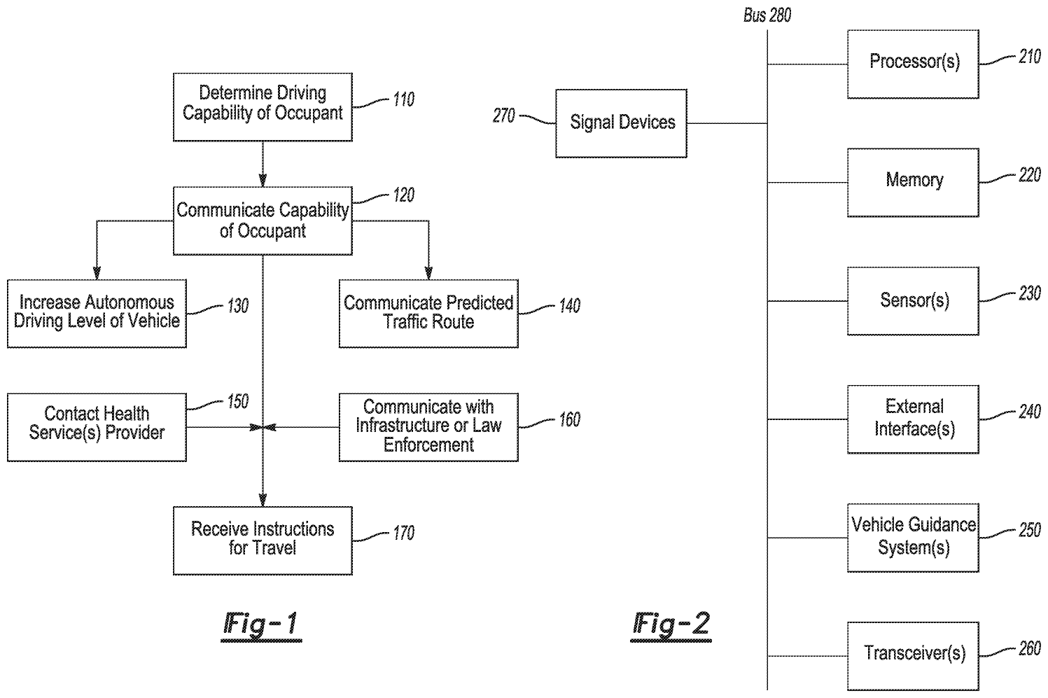

FIG. 1 illustrates a method according to one or more embodiments. As shown in FIG. 1, the method can include, at 110, determining a driving capability of an occupant of a vehicle, such as the driver of the vehicle or other occupant of the vehicle, while the vehicle is driving. In a fully autonomous vehicle, there may or may not be a designated driver. Accordingly, in one or more embodiments any vehicle occupant of a fully autonomous vehicle can be considered a driver, or person of interest within the vehicle. In other embodiments, the vehicle may have a human driver, but that human may not be actively driving the vehicle when the vehicle is a fully autonomous mode (e.g., the human, or person, may be positioned in a driver's seat and capable of driving, while the vehicle to operates autonomously). Likewise, a person who was driving the vehicle may lose consciousness or otherwise encounter a physical or mental state that prevents the person from driving the vehicle. Nevertheless, in one or more embodiments this person can be designated as the driver on a continuing basis, despite the fact that the person may no longer be actively involved in the steering or other controls of the vehicle.

The method can also include, at 120, communicating an indicator of the capability of the driver (or other occupant) from the vehicle. There are two aspects of one or more embodiments: (1) capability of the driver to operate the vehicle and (2) health issue of any occupant within the vehicle. The indicator of the capability of the driver (or other occupant) can broadly refer to both or either of these aspects. Thus, in one or more embodiments, there can be a determination of a driving capability of a driver of a vehicle while the driver is driving and a communication of an indicator of the capability of the driver from the vehicle to an entity outside of the driving vehicle.

The driving capability can include an attentiveness of the driver or a sleep state of a driver. The determining can include monitoring eye tracking, monitoring facial expressions, monitoring consciousness, monitoring pain, monitoring steering departures, monitoring an output of a heart rate monitor, monitoring an output of an electrocardiogram, monitoring blood pressure, monitoring driver posture, or any combination thereof.

The communicating can be done individually to one or more entities outside the vehicle, such as a remotely located computing device. Alternatively, or in addition, the communicating can be involve broadcasting or multicasting the capability. The communicating can involve communicating to a plurality of nearby vehicles. The communicating can use vehicle to X, WiFi, or another wireless communication technology. Thus, the communicating can involve peer-to-peer communicating. The communicating can be addressable, to contact a specific recipient, group, or class of recipients. For example, the class of emergency responders can be a designated recipient.

The communicating can include communicating travel information regarding the vehicle. The travel information can include speed of the vehicle, direction of the vehicle, acceleration of the vehicle, geographic location of the vehicle, or any combination of thereof. The communicating can further include communicating an autonomous mode status of the vehicle.

The communicating can include contacting an emergency responder. Thus, for example, the entity outside the vehicle can include other regular vehicles and additionally an emergency responder, or both regular vehicles and an emergency responder.

The method can include, at 130, increasing an autonomous driving level of the vehicle based on the determination of the driving capability of the driver.

The method can further include, at 140, communicating a predicted traffic route of the vehicle. This route can be communicated to surrounding vehicles and/or emergency responders. The communicating can further include contacting a system configured to coordinate traffic signals. Thus, for example, the vehicle can be configured to request lights to be green on the predicted traffic route to expedite delivery of the driver or other occupant to an emergency responder.

The method can additionally include, at 150, contacting a health services provider regarding the driver. This health services provider may be, for example, an emergency room, urgent care clinic, doctor's office, or other inpatient or outpatient medical care center. Thus, for example, the contacting can include informing an emergency room of a projected arrival time of the driver. The contacting can include coordinating an exchange point with an ambulance to deliver the driver to the ambulance. Thus, this contacting can also include, in one or more embodiments, the communicating the predicted traffic route at 140 (for example, the predicted traffic route of the vehicle can be communicated to a health service provider, thereby performing both 140 and 150 in a single communication). Additionally, the contacting the health services provider can include health information of the driver to the health services provider. This health information can include symptoms, onset of the symptoms, and severity of the symptoms. The transfer of health information can be approved in advance of the situation by a configuration set by the driver or other occupant or by that person's legal guardian or legal representative.

In one or more embodiments, for example, the vehicle may be configured to handle highway driving autonomously, but may not be as safe at other kinds of driving. Accordingly, the vehicle may select a rendezvous point with an emergency vehicle or other emergency responder at, for example, an exit of a highway. Other scenarios are also permitted.

The health information can include eye movement data, pain data, facial expression data, consciousness data, heart rate data, blood pressure data, electrocardiogram data, posture data, or any combination thereof. The driver can select which categories, types, or specific values of data can be communicated through a configuration, which may occur through a vehicle interface or a user equipment, such as a smart phone or smart watch.

The communicating can include emitting an audible alert, a visual alert, or a combination thereof, from the vehicle. This may enable the vehicle to be more clearly identified as a vehicle in an emergency. For example, the visual alert may include toggling between headlights or high beams on the headlights. The audible alert may include honking the vehicle's horn or emitting a siren or other warning sound.

The method can additionally include, at 160, communicating with an infrastructure system or law enforcement system to request permission to change traffic controls or break traffic controls. The changing the traffic controls can include changing a color of a traffic light, for example, from red to green or from flashing red to flashing yellow. Other changes are also permitted. Breaking the traffic controls can include exceeding a posted speed limit.

The method can also include, at 170, receiving, in response to the indicator, instructions to proceed to a target route or destination, such as a medical facility. The instructions can include instructions for the vehicle to autonomously navigate or remote control instructions. The remote control instructions can be provided based on the vehicle being within a predetermined distance or within a predetermined number of turns from the medical facility. In certain cases, a user interface at the medical facility may permit a remote driver to control the vehicle to the medical facility.

FIG. 2 illustrates a system according to one or more embodiments. The system illustrated in FIG. 2 may be embodied in a vehicle or in one or more components of a vehicle. For example, one or more embodiments may be implemented as an electronic control unit (ECU) of a vehicle.

The system can include, one more processors 210 and one more memories 220. The processor 210 and memory 220 can be embodied on a same chip, on different chips, or otherwise separate or integrated with one another. The memory 220 can be a non-transitory computer-readable memory. The memory 220 can contain a set of computer instructions, such a computer program. The computer instructions, when executed by the processor 210, can perform a process, such as the method shown in FIG. 1, or any of the other methods disclosed herein.

The processor 210 may be one or more computer chips including one or more processing cores. The processor 210 may be an application specific integrated circuit (ASIC) or a field programmable gate array (FPGA). The memory 220 can be a random access memory (RAM) or a read only memory (ROM). The memory 220 can be a magnetic medium, an optical medium, or any other medium.

The system can also include one or more sensors 230. The sensors 230 can include devices that monitor a condition of a vehicle occupant, such a driver. Devices can include, for example, heart rate sensors, electrocardiogram (EKG) devices, visible light cameras, infrared cameras, gesture detection devices, and the like.

The system can also one or more external interfaces 240. The external interface 240 can be a wired or wireless connection to a device that is not itself a component of the vehicle. Such devices may include, for example, smart phones, smart watches, personal digital assistants, smart pedometers, fitness wearable devices, smart medical devices, or any other portable or wearable electronics.

The system can also include one or more vehicle guidance systems 250. The vehicle guidance system 250 may include its own sensors, interfaces, and communication hardware. For example, the vehicle guidance system 250 may be configured to permit fully autonomous, semi-autonomous, and manual driving. The vehicle guidance system 250 may be able to assume steering control, throttle control, traction control, braking control, and other control from a human driver. The vehicle guidance system 250 may be configured to operate in conjunction with an advanced driver assistance system, which can have features such as automatic lighting, adaptive cruise control and collision avoidance, pedestrian crash avoidance mitigation (PCAM), satnav/traffic warnings, lane departure warnings, automatic lane centering, automatic braking, and blind-spot mitigation.

The system can further include one or more transceivers 260. The transceiver 260 can be a WiFi transceiver, a V2X transceiver, or any other kind of wireless transceiver, such as a satellite or cellular communications transceiver.

The system can further include signal devices 270. The signal device 270 may be configured to provide an audible warning (such as a siren or honking noise) using, for example, an acoustic device or a visual warning (such as flashing or strobing lights) using, for example, an illumination device (e.g., light or other suitable device). The signal device 270 may be provided by a vehicle's horn and/or headlights and taillights. Other signals are also permitted. In some embodiments, the system may determine whether the capability (e.g., as determined) is above a threshold. When the system determines that the capability is above the threshold, the system may generate the audible warning using the acoustic device and/or generate the visual warning using the illumination device.

The signal device 270, transceiver 260, vehicle guidance system 250, external interface 240, sensor 230, memory 220, and processor 210 may be variously communicably connected, such as via a bus 280, as shown in FIG. 2. Other topologies are permitted. For example, the use of a car area network (CAN) is permitted.

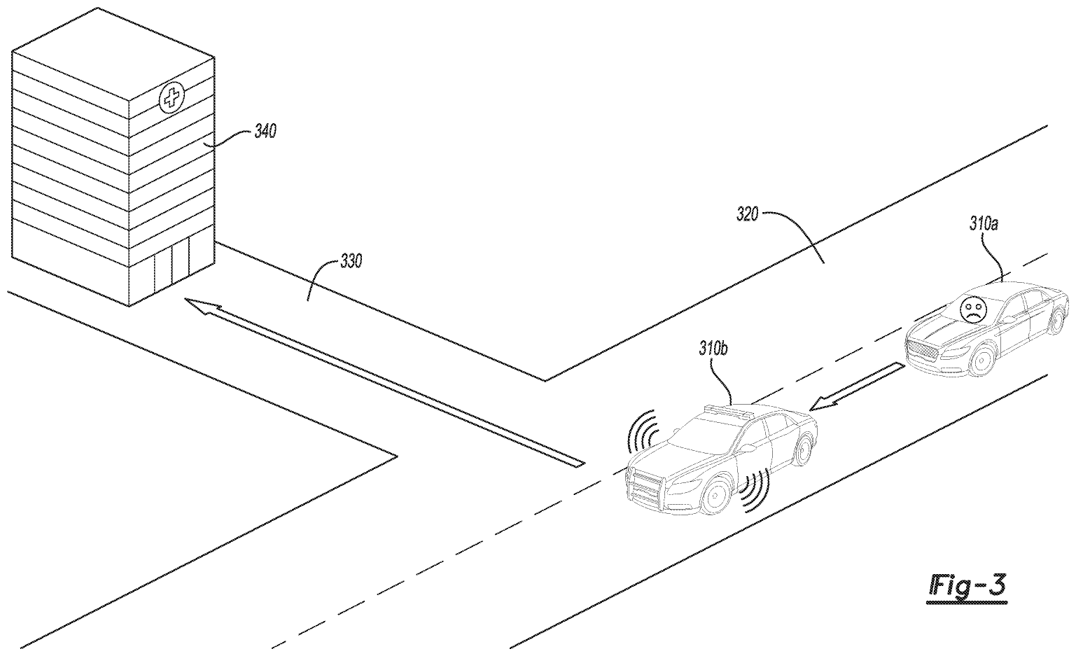

FIG. 3 generally illustrates a scenario according to one or more embodiments. As shown in FIG. 3, a vehicle at a first time 310a may detect a health issue, such as a health anomaly, or other capability issue, as described above. The health issue may be of a driver or other person in the vehicle. For example, as described, a driver of the vehicle may experience a health issue that makes driving impractical or impossible. Additionally, or alternatively, another person within the vehicle may experience the health issue. This can be considered a distress detection. Thereafter, the same vehicle at a second time 310b may send a distress message (for example, by broadcast or addressed communication). The vehicle may also convert itself to an emergency vehicle mode, as described above. For example, the system, as described, may be configured to determine whether the health issue requires emergency medical attention. In the case of the driver experiencing the health issue, the system may determine if the health issue is above a first threshold (e.g., a health condition that renders the driver incapable of driving). When the system determines that the health issue of the driver is above the threshold, the system may convert the vehicle to the emergency vehicle mode. In another example, when the other person in the vehicle (e.g., not the driver) experiences the health issue, the system may determined whether the health issue is above a second threshold (e.g., greater than the first threshold. For example, the system may determine whether the other person in the vehicle needs immediate medical attention. When the system determines that the health issue is above the second threshold, the system may convert the vehicle to the emergency vehicle mode. When the health issue is below the second threshold, the system may allow the driver to operate the vehicle in order to seek medical treatment for the other person as necessary. The vehicle may depart from road 320 to a new road 330 to proceed to a medical facility 340. The vehicle at 310b may communicate with the medical facility 340 as described above.

In some embodiments, the vehicle may be part of a ride sharing system. For example, users may request a ride using an application on a smart phone or other computing device. The vehicle may arrive and transport the user to a desired location. When the vehicle is operated by a driver, the system may continue as described in the event that the driver of the ride sharing vehicle or another person in the ride sharing vehicle experiences a health issue. Additionally, or alternatively, the vehicle may include an autonomous ride sharing vehicle. The autonomous ride sharing vehicle may transport users, as described, and may convert to an emergency vehicle mode in response to a user within the vehicle experiencing a health issue. Additionally, or alternatively, a user may require medical assistance and may, using the application as described, request the vehicle for transportation as an emergency vehicle. For example, the vehicle may be operating in a mode other than the emergency vehicle mode (e.g., either at rest or being driven, autonomously or otherwise), the user may experience a health issue and require medical assistance. The user may use the application to request a ride from the vehicle. The vehicle may convert to the emergency vehicle mode and collect the user for transportation.

In some embodiments, an apparatus includes at least one processor; and at least one memory including computer program code. The at least one memory and the computer program code are configured to, with the at least one processor, cause the apparatus at least to determine a capability of an occupant of a vehicle while driving; and communicate an indicator from the vehicle.

In some embodiments, the capability comprises a driving capability of a driver of the vehicle to operate the vehicle or a health issue of the occupant within the vehicle. In some embodiments, the communicating comprises broadcasting, multicasting, or peer-to-peer communicating the capability. In some embodiments, the communicating comprises communicating to a single addressed nearby vehicle or a plurality of nearby vehicles. In some embodiments, the communicating comprises communicating using vehicle to X, WiFi, or another wireless communication technology. In some embodiments, the communicating further comprises communicating travel information regarding the vehicle. In some embodiments, the travel information comprises speed of the vehicle, direction of the vehicle, acceleration of the vehicle, geographic location of the vehicle, or any combination of thereof. In some embodiments, the communicating further comprises communicating an autonomous mode status of the vehicle. In some embodiments, the communicating comprise contacting an emergency responder. In some embodiments, the at least one memory and the computer program code are configured to, with the at least one processor, cause the apparatus at least to increase an autonomous driving level of the vehicle based on the determination of the driving capability of the driver. In some embodiments, the at least one memory and the computer program code are configured to, with the at least one processor, cause the apparatus at least to communicate a predicted traffic route of the vehicle. In some embodiments, the communicating comprises contacting a system configured to coordinate traffic signals. In some embodiments, the at least one memory and the computer program code are configured to, with the at least one processor, cause the apparatus at least to contact a health services provider regarding the driver. In some embodiments, the contacting comprises informing an emergency room of a projected arrival time of the driver. In some embodiments, the contacting comprises coordinating an exchange point with an ambulance to deliver the driver to the ambulance. In some embodiments, the contacting comprises communicating health information of the driver to the health services provider. In some embodiments, the health information comprises eye movement data, pain data, facial expression data, consciousness data, heart rate data, blood pressure data, electrocardiogram data, posture data, or any combination thereof. In some embodiments, the communicating comprises emitting an audible alert, a visual alert, or a combination thereof, from the vehicle. In some embodiments, the at least one memory and the computer program code are configured to, with the at least one processor, cause the apparatus at least to communicate with an infrastructure system or law enforcement system to request permission to change traffic controls or break traffic controls. In some embodiments, changing the traffic controls comprises changing a color of a traffic light. In some embodiments, breaking the traffic controls comprises exceeding a posted speed limit. In some embodiments, the at least one memory and the computer program code are configured to, with the at least one processor, cause the apparatus at least to receive, in response to the indicator, instructions to proceed to a medical facility. In some embodiments, the instructions comprise instructions for the vehicle to autonomously navigate or remote control instructions. In some embodiments, the remote control instructions are provided based on the vehicle being within a predetermined distance or within a predetermined number of turns from the medical facility. In some embodiments, the apparatus is configured to perform an autonomous safe-stop of the vehicle responsive to the determined capability. In some embodiments, the apparatus is configured to communicate with autonomous vehicles in a path of the vehicle to clear a path responsive to the determined capability. In some embodiments, the apparatus is configured to communicate with cyclists, human drivers, or pedestrians in a path of the vehicle to clear a path responsive to the determined capability. In some embodiments, the apparatus is configured to communicate the indicator to other vehicles in autonomous driving mode, the indicator comprising information configured to prepare the other vehicles to predict or otherwise calculate a way to navigate away from a predicted travel path of the vehicle. In some embodiments, the communicating the indicator comprises communicating a travel path of the vehicle and a projected travel time of the vehicle to a destination emergency center or hospital. In some embodiments, the communicating the indicator further comprises communicating a request for a route for navigating the vehicle to a medical facility. In some embodiments, the communicating the indicator further comprises requesting identification of a road entrance to use, a drop off point to use, or both for the occupant. In some embodiments, the apparatus is further configured to receive, responsive to the request, one or more commands to the vehicle to reduce an immediate health issue of the occupant or make the occupant more comfortable.

In some embodiments, an apparatus includes at least one processor; and at least one memory including computer program code. The at least one memory and the computer program code are configured to, with the at least one processor, cause the apparatus at least to receive, at a vehicle, an indicator of a capability of an occupant of a further vehicle; and at least one of display a warning within the vehicle based on the indicator or relay the indicator.

In some embodiments, a system for a vehicle includes a processor and a memory including computer program code. The memory and the computer program code are configured to, with the processor, cause the system to: determine a capability of a person; and communicate an indicator, from the system to a remote device, based on the capability of the person.

In some embodiments, the capability includes a driving capability of the person. In some embodiments, the capability includes a health issue of the person. In some embodiments, the system is configured to wirelessly communicate the indicator to the remote device. In some embodiments, the indicator includes travel information regarding the vehicle, the travel information includes a speed of the vehicle, a direction of the vehicle, an acceleration of the vehicle, a geographic location of the vehicle, or a combination thereof. In some embodiments, the indicator includes an autonomous mode status of the vehicle.

In some embodiments, a system for a vehicle includes a sensor and a processor. The sensor is configured to monitor an occupant of the vehicle and the processor is coupled to the sensor and configured to: determine, based on the sensor, a condition of the occupant of the vehicle; determine, based on the condition, a capability of the occupant; and communicate an indicator, from the system to a remote device remotely located from the vehicle, based on the capability of the occupant.

In some embodiments, the capability includes a driving capability of the occupant. In some embodiments, based on a determination that the driving capability is below a threshold value, the processor is configured to operate the vehicle in an autonomous mode of operation. In some embodiments, based on a determination that the capability includes a health issue of the occupant, the processor is configured to operate the vehicle in an autonomous mode of operation. In some embodiments, the system also includes a transceiver coupled to the processor, wherein the processor is configured to wirelessly communicate the indicator, via the transceiver, to the remote device. In some embodiments, the indicator includes travel information regarding the vehicle, the travel information includes a speed of the vehicle, a direction of the vehicle, an acceleration of the vehicle, a geographic location of the vehicle, or a combination of thereof. In some embodiments, the indicator includes an autonomous mode status of the vehicle. In some embodiments, the system also includes an acoustic device coupled to the processor for generating an audible warning. In some embodiments, the processor is configured to generate the audible warning using the acoustic device, based on a determination that the capability of the occupant is below a threshold value. In some embodiments, the system also includes an illumination device coupled to the processor for generating a visual warning. In some embodiments, the processor is configured to generate the visual warning using the illumination device, based on a determination that the capability of the occupant is below a threshold value.

In some embodiments, a non-transitory computer-readable medium includes instructions that, when executed by a processor, cause the processor to operate a vehicle in an autonomous mode of operation, by performing the steps of: monitoring, via a sensor, an occupant of the vehicle; determining, based on the monitoring of the occupant, a condition of the occupant; determining, based on the condition of the occupant, a capability of the occupant; and operating the vehicle in the autonomous mode of operation, based on a determination that the capability of the occupant is below a threshold value.

In some embodiments, the steps further include wirelessly communicating an indicator to a remote device, based on the determination that the capability of the occupant is below the threshold value. In some embodiments, the indicator includes an autonomous mode status of the vehicle.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

The word “example” is used herein to mean serving as an example, instance, or illustration. Any aspect or design described herein as “example” is not necessarily to be construed as preferred or advantageous over other aspects or designs. Rather, use of the word “example” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an implementation” or “one implementation” throughout is not intended to mean the same embodiment or implementation unless described as such.

Implementations the systems, algorithms, methods, instructions, etc., described herein can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

As used herein, the term module can include a packaged functional hardware unit designed for use with other components, a set of instructions executable by a controller (e.g., a processor executing software or firmware), processing circuitry configured to perform a particular function, and a self-contained hardware or software component that interfaces with a larger system. For example, a module can include an application specific integrated circuit (ASIC), a Field Programmable Gate Array (FPGA), a circuit, digital logic circuit, an analog circuit, a combination of discrete circuits, gates, and other types of hardware or combination thereof. In other embodiments, a module can include memory that stores instructions executable by a controller to implement a feature of the module.

Further, in one aspect, for example, systems described herein can be implemented using a general-purpose computer or general-purpose processor with a computer program that, when executed, carries out any of the respective methods, algorithms, and/or instructions described herein. In addition, or alternatively, for example, a special purpose computer/processor can be utilized which can contain other hardware for carrying out any of the methods, algorithms, or instructions described herein.

Further, all or a portion of implementations of the present disclosure can take the form of a computer program product accessible from, for example, a computer-usable or computer-readable medium. A computer-usable or computer-readable medium can be any device that can, for example, tangibly contain, store, communicate, or transport the program for use by or in connection with any processor. The medium can be, for example, an electronic, magnetic, optical, electromagnetic, or a semiconductor device. Other suitable mediums are also available.

The above-described embodiments, implementations, and aspects have been described in order to allow easy understanding of the present invention and do not limit the present invention. On the contrary, the invention is intended to cover various modifications and equivalent arrangements included within the scope of the claims, which scope is to be accorded the broadest interpretation to encompass all such modifications and equivalent structure as is permitted under the law.

What is claimed is:

1. A system for a vehicle, the system comprising:
a processor; and
a memory including computer program code,
wherein the memory and the computer program code are configured to, with the processor, cause the system to:
determine a capability of a person; and
communicate an indicator , from the system to a remote device, based on the capability of the person.

2. The system of claim 1, wherein the capability includes a driving capability of the person.

3. The system of claim 1, wherein the capability includes a health issue of the person.

4. The system of claim 1, wherein the system is configured to wirelessly communicate the indicator to the remote device.

5. The system of claim 1, wherein the indicator includes travel information regarding the vehicle, the travel information includes a speed of the vehicle, a direction of the vehicle, an acceleration of the vehicle, a geographic location of the vehicle, or a combination thereof.

6. The system of claim 1, wherein the indicator includes an autonomous mode status of the vehicle.

7. A system for a vehicle, the system comprising:
a sensor configured to monitor an occupant of the vehicle; and
a processor coupled to the sensor and configured to:
determine, based on the sensor, a condition of the occupant of the vehicle;
determine, based on the condition, a capability of the occupant; and
communicate an indicator, from the system to a remote device remotely located from the vehicle, based on the capability of the occupant.

8. The system of claim 7, wherein the capability includes a driving capability of the occupant.

9. The system of claim 8, wherein, based on a determination that the driving capability is below a threshold value, the processor is configured to operate the vehicle in an autonomous mode of operation.

10. The system of claim 7, wherein, based on a determination that the capability includes a health issue of the occupant, the processor is configured to operate the vehicle in an autonomous mode of operation.

11. The system of claim 7, further comprising a transceiver coupled to the processor, wherein the processor is configured to wirelessly communicate the indicator, via the transceiver, to the remote device.

12. The system of claim 7, wherein the indicator includes travel information regarding the vehicle, the travel information includes a speed of the vehicle, a direction of the vehicle, an acceleration of the vehicle, a geographic location of the vehicle, or a combination of thereof.

13. The system of claim 12, wherein the indicator includes an autonomous mode status of the vehicle.

14. The system of claim 7, further comprising an acoustic device coupled to the processor for generating an audible warning.

15. The system of claim 14, wherein the processor is configured to generate the audible warning using the acoustic device, based on a determination that the capability of the occupant is below a threshold value.

16. The system of claim 7, further comprising an illumination device coupled to the processor for generating a visual warning.

17. The system of claim 16, wherein the processor is configured to generate the visual warning using the illumination device, based on a determination that the capability of the occupant is below a threshold value.

18. A non-transitory computer-readable medium including instructions that, when executed by a processor, cause the processor to operate a vehicle in an autonomous mode of operation, by performing steps, including:
monitoring, via a sensor, an occupant of the vehicle;
determining, based on the monitoring of the occupant, a condition of the occupant;
determining, based on the condition of the occupant, a capability of the occupant; and
operating the vehicle in the autonomous mode of operation, based on a determination that the capability of the occupant is below a threshold value.

19. The non-transitory computer-readable medium of claim 18, wherein the steps further include wirelessly communicating an indicator to a remote device, based on the determination that the capability of the occupant is below the threshold value.

20. The non-transitory computer-readable medium of claim 19, wherein the indicator includes an autonomous mode status of the vehicle.
